(19) United States
(12) Patent Application Publication
(10) Pub. No.: US 2013/0211515 A1
(43) Pub. Date: Aug. 15, 2013

(54) IMPLANTABLE OPHTHALMIC DEVICES WITH CIRCULARLY ASYMMETRIC OPTIC AND METHODS

(71) Applicants: Ronald D. Blum, Roanoke, VA (US); Amitava Gupta, Roanoke, VA (US); Julien Sauvet, Liebefeld-Bern (CH); Jean-Noel Fehr, Liebefeld-Bern (CH); Urban Schnell, Liebefeld-Bern (CH)

(72) Inventors: Ronald D. Blum, Roanoke, VA (US); Amitava Gupta, Roanoke, VA (US); Julien Sauvet, Liebefeld-Bern (CH); Jean-Noel Fehr, Liebefeld-Bern (CH); Urban Schnell, Liebefeld-Bern (CH)

(73) Assignee: Elenza, Inc

(21) Appl. No.: 13/805,742

(22) PCT Filed: Jun. 24, 2011
(86) PCT No.: PCT/US11/41764

§ 371 Date: Mar. 4, 2013

Related U.S. Application Data
(60) Provisional application No. 61/358,569, filed on Jun. 25, 2010.

(51) Int. Cl. A61F 2/16

(52) U.S. Cl. CPC A61F 2/1624
USPC 623/6.22

(57) ABSTRACT

Astigmatism is an optical aberration that displaces the eye's vertical focal plane with respect to its horizontal focal plane. This displacement in focal planes, which may be caused by an irregularly shaped cornea and/or crystalline lens, causes images to appear blurry. Astigmatism can be corrected by implanting an optic, such as section of a spherical lens, whose projection onto a plane perpendicular to the optical axis is noncircular (e.g., rectangular or elliptical). Because the optic is noncircular, it provides more optical power along one axis than along another axis. As a result, it introduces an astigmatism that can be used to offset or compensate the eye's corneal and/or lenticular astigmatism when aligned properly with respect to the principal meridians of the cornea and/or crystalline lens.

### CROSS-REFERENCE TO RELATED PATENT APPLICATIONS

This application claims the benefit of U.S. Provisional Application No. 61/358,569 filed Jun. 25, 2010, and entitled “Use of Non Circular Optical Implants to Correct Aberrations in the Eye.” The above-referenced application is incorporated herein by reference in its entirety

### BACKGROUND

An astigmatic eye focuses light to two or more focal planes instead of to a single focal plane (i.e, the retina). As a result, an off-axis point object may appear as a vertical line in one focal plane and as a horizontal line in another focal plane, with a circle of least confusion appearing between the focal planes. Astigmatism usually blurs and/or distorts images of objects at all distances to some degree and results in eye strain, squinting, and headaches, especially after reading. In most cases, astigmatism is caused by a cornea that is shaped like an ellipsoid instead of a sphere: the cornea has a different radius of curvature around different meridians, with the largest and smallest radii of curvature lying along what are known as the principal meridians. (Astigmatism can also be caused by an irregularly shaped crystalline lens.)

Astigmatism can be classified based on the orientation of the principal meridians. In regular corneal astigmatism, the principal meridians are perpendicular to each other, and may be classified as with-the-rule, against-the-rule, or oblique depending on their exact orientation. In irregular corneal astigmatism, the principal meridians are not perpendicular to each other.

Astigmatism can also be classified based on the locations of the focal lines of the principal meridians with respect to the retina. In simple astigmatism, one focal line is on the retina, and the other focal line is either behind the retina (hyperopic) or in front of the retina (myopic). In compound astigmatism, both focal lines are either behind the retina (hyperopic) or in front of the retina (myopic). In mixed astigmatism, the focal lines straddle the retina.

Astigmatism affects a large portion of population. A recent study of American children found that 28 percent had an astigmatism of at least 1.0 diopter. A study conducted recently in the United Kingdom found that 47.4 percent of more than 11,000 eyeglass wearers had astigmatism of 0.75 D or greater in at least one eye; 24.1 percent had this amount of astigmatism in both eyes. Myopic astigmatism (31.7 percent) occurred about twice as often as hyperopic astigmatism (15.7 percent). Astigmatism, like nearsightedness and farsightedness, can usually be corrected with eyeglasses, contact lenses, or refractive surgery. However, eyeglasses detract from one's natural appearance, contact lenses must be replaced on a regular basis, and refractive surgery can lead to a host of complications, including halos, doubling of vision, light scattering, glare, loss of contrast sensitivity, limited range of focus, and/or reduction of light hitting the retina.

### SUMMARY

Embodiments of the technology disclosed herein include an implantable ophthalmic device with a optic that defines an optical axis. The optic, which can be a lens element, has a surface that is perpendicular to the optical axis and bounded by a perimeter that lacks circular symmetry. The optic can have an astigmatism of about 0.50 diopters to about 2.0 diopters. The device may include at least one lens anchor coupled to the optic and configured to maintain the optic in a stable position when implanted in a patient's eye.

In at least one illustrative embodiment, the optic has at least one major axis perpendicular to the optical axis and at least one minor axis perpendicular to the major axis. The major axis can have a length of about 4 mm to about 7 mm, and the device may be implanted in an eye with corneal astigmatism such that the major axis is orthogonal to an axis of the corneal astigmatism.

The optic may have a graded index profile, or, alternatively, the surface of the optic may be a focusing surface, such as a spherical or aspheric surface. In some cases, the optic may have a (compensatory) spherical aberration. An exemplary spherical surface may have a radius of curvature of about 15 mm to about 100 mm.

In some examples, the perimeter of the surface of the optic includes a plurality of edges. These edges may be arranged to define a convex polygon, such as a rectangle, which may have a ratio of width to length of about 1.2 to about 3.5. Alternatively, the perimeter may form an ellipse whose major axis may about 5.0 mm to about 7.0 mm long and whose minor axis may be about 2.0 mm to about 6.0 mm long.

Other embodiments include an implantable ophthalmic device (and method of operating such a device) with an electro-active cell configured to form a noncircular aperture, in a plane normal to the optical axis, whose major axis is aligned with respect to a principal meridian of an astigmatic eye. Actuating the electro-cell to form the noncircular aperture can introduce an astigmatism of about 0.10 diopters to about 2.0 diopters. The major (long) axis of the aperture can be aligned such that it is orthogonal to the principal meridian of the astigmatic eye. The major axis can have a length that is about 1.2 to about 3.5 times larger than the shortest dimension of the noncircular aperture.

To implant an illustrative implantable ophthalmic device with a optic bounded by a noncircular perimeter, one makes a mark on the scelera of the eye indicative of an astigmatic characteristic of the eye. Next, one inserts the device into the eye and aligns the noncircular perimeter of the optic with respect to the mark on the scelera. The device may be secured by deploying one or more lens anchors coupled to the optic after the perimeter of the optic is aligned with respect to the mark on scelera. In some cases, one measures the astigmatic characteristic of the eye before marking the scelera.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

The accompanying drawings, which are incorporated in and constitute a part of this specification, illustrate embodiments of the invention and together with the description serve to explain principles of the invention.

FIG. 1 shows a cross section of a healthy (non-astigmatic) human eye.

FIGS. 2A-2C shows plan, elevation, and perspective views of an illustrative implantable ophthalmic device with a noncircular lens element.

FIGS. 3A-3C shows plan views of alternative implantable ophthalmic devices with noncircular lens elements.

FIG. 4 is a schematic diagram that illustrates how an aperture can be used with a conventional lens to provide a lens element with a noncircular perimeter.

FIGS. 5A-5C shows plan, elevation, and perspective views of an electro-active element used to mask a circular lens to provide a noncircular lens element.

FIG. 6 is a ray diagram that illustrates (left) a model of a perfect eye imaging an object at infinity and (right) an electro-active cell used to provide a noncircular aperture that introduces astigmatism.

FIG. 7 includes plots of polychromatic modulation transfer functions (MTFs) for the perfect eye model at pupil diameters of 3 mm (left) and 5 mm (right).

FIG. 8 includes plots of polychromatic MTFs for the perfect eye model with an exemplary noncircular aperture tilted at 0° and a pupil diameter of 3 mm (left) and tilted at 45° and a pupil diameter of 5 mm (right).

FIG. 9 is a plot of MTFs calculated using the perfect eye model and a 5 mm pupil diameter for: the diffraction limit (top line); the perfect eye itself (upper middle line); an illustrative noncircular aperture tilted at 45° (lower middle line); and sagittal and tangential ray fans and an illustrative implantable noncircular aperture tilted at 0° (bottom line).

FIG. 10 is a ray diagram of a lens and aperture used in a Liou and Brennan model of an astigmatic eye.

FIG. 11 includes plots of sagittal and tangential polychromatic MTFs for a Liou and Brennan eye model with pupil diameters of 3 mm (left) and 5 mm (right).

FIG. 12 includes plots of polychromatic MTFs for a Liou and Brennan eye model with an exemplary implantable noncircular aperture tilted at 0° and a pupil diameter of 3 mm (left) and tilted at 45° and a pupil diameter of 5 mm (right).

FIG. 13 is a plot of sagittal MTFs (dashed lines) and tangential MTFs calculated using the Liou and Brennan eye model and a 5 mm pupil diameter for no implant and for illustrative implantable noncircular apertures tilted at 0°, 45°, and 90°.

FIG. 14 is a plot of on-retina astigmatism versus aperture eccentricity calculated using the Liou and Brennan eye model.

### DETAILED DESCRIPTION

Presently preferred embodiments of the invention are illustrated in the drawings. An effort has been made to use the same or like reference numbers to refer to the same or like parts.

The Eye

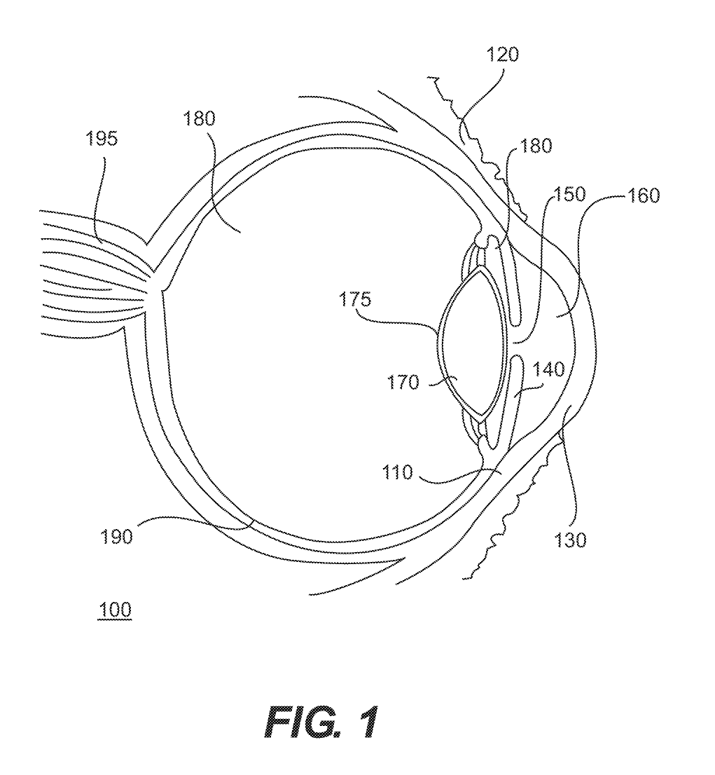

FIG. 1 shows a cross section of a healthy human eye 100. The white portion of the eye is known as the sclera 110 and is covered with a clear membrane known as the conjunctiva 120. The central, transparent portion of the eye that provides most of the eye's optical power is the cornea 130. The iris 140 is the pigmented portion of the eye 100 and forms the pupil 150. The sphincter muscles constrict the pupil 150 and the dilator muscles dilate the pupil. The pupil 150 is the natural aperture of the eye 100. The anterior chamber 160 is the fluid-filled space between the iris and the innermost surface of the cornea. The crystalline lens 170 is held in the lens capsule 175 and provides the remainder of the eye's optical power. The retina 190, which is separated from the back surface of the iris 140 by the posterior chamber 180, acts as the “image plane” of the eye 100 and is connected to the optic nerve 195, which conveys visual information to the brain.

In individuals suffering from astigmatism, the cornea 130 and/or the crystalline lens 170 are irregularly shaped (i.e., they lack circular symmetry about the eye's optical axis), which results in an undesired variation in optical power as a function meridian angle. In other words, astigmatic eyes do not behave as spherical lenses; they behave as spherical lenses with additional cylindrical power. As a result, an astigmatic eye does not project sharp images onto the retina.

It is known that astigmatism in the eye appears mainly in the cornea and the crystalline lens. Since the cataractous crystalline lens is surgically removed during cataract surgery and usually replaced by a non toric intraocular lens, the sole contributor to astigmatism in a pseudophakic eye is the cornea. The cornea of a pseudophakic eye may have preexisting astigmatism as well as astigmatism induced by tension developing in the corneal tissue caused by the healing process that occurs over a period of several months following surgery. The size, shape and orientation of the incision or incisions made on the corneal tissue control the orientation and magnitude of the surgically induced astigmatism. Some cataract surgeons choose to provide compensation of preexisting astigmatism in the cornea by inducing orthogonal astigmatism in the cornea through astigmatic keratectomy. The inherent uncertainty in the healing process may cause the induced astigmatism to be different in amplitude and direction that that was planned, and consequently lead to the development of irregular astigmatism following such a procedure. A second method to correct preexisting astigmatism in the cataractous eye involves the use of a toric intraocular lens that provides a toric correction. A toric intraocular lens is designed with a toric optic that features two different curvatures along orthogonal meridians of the optic.

Noncircular Implantable Ophthalmic Devices

Illustrative embodiments of the implantable ophthalmic devices disclosed herein provide astigmatic lens elements that can be used to treat astigmatism. In general, an illustrative implantable ophthalmic device has an optic with a surface that is orthogonal to the device's optical axis. (In general, the optical axis of an optical element or optical system is defined as the line about which the optical element or optical system possesses some degree of rotational symmetry.) The optic has a focusing surface or graded index profile that is bounded by a perimeter that, unlike in a conventional spherical or aspheric lens, is not a circle—rather, the optic has a noncircular cross section in a plane perpendicular to its optical axis. In other words, the optic's perimeter lacks circular symmetry about the optical axis, i.e., its appearance varies with different degrees of rotation about the optical axis.

This lack of circular symmetry leaves the device with at least one major axis and at least one minor axis, both of which are perpendicular to the optical axis. In some cases (e.g., for rectangular and elliptical perimeters), the major and minor axes may be perpendicular to each other as well. Because the focusing surface or graded index profile does not extend by equal amounts along its major and minor axes, the illustrative implantable ophthalmic device has different amounts of optical power along its major and minor axes. As a result, the device is astigmatic by an amount that depends on the relative lengths of the major and minor axes and the base optical power of the optic. This astigmatism can be selected such that it compensates corneal or lenticular astigmatism when implanted in an eye.

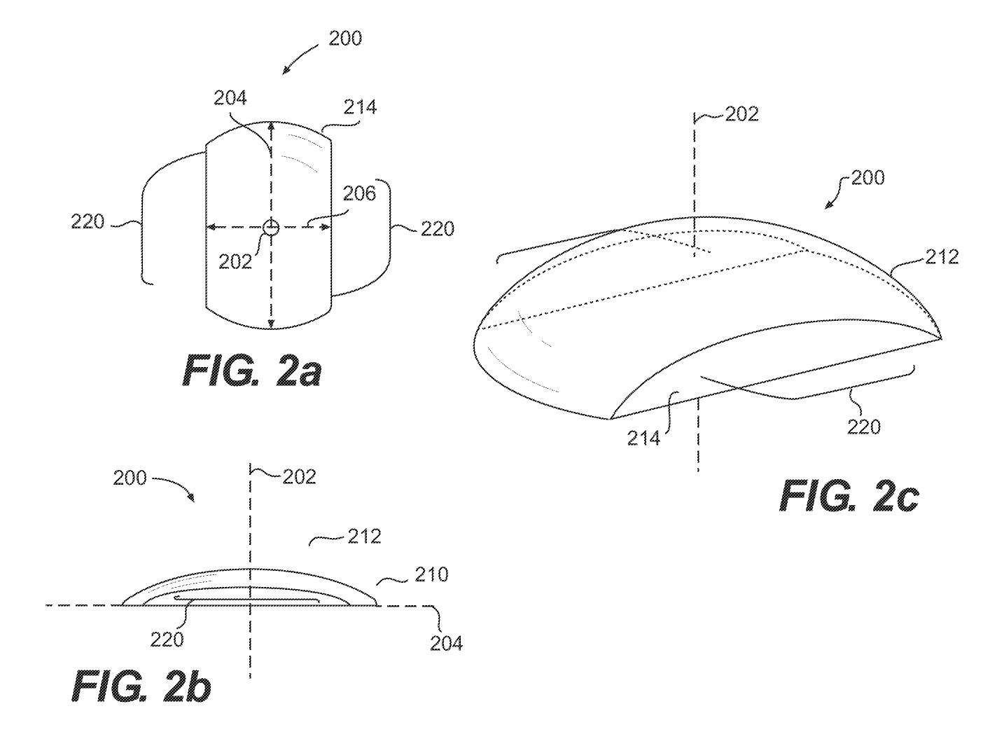

FIGS. 2A-2C shows plan, elevation, and perspective views of an exemplary noncircular implantable ophthalmic device 200 that includes lens anchors (haptics) 220 coupled to the sides of a lens element 210. The optic 210 has a focusing surface 212 that is normal to the device's optical axis 202. (Although the optic 210 shown in FIGS. 2A-2C is a plano-convex lens element, those of skill in the art will readily appreciate that the optic could also be a diffractive element, holographic element, prism, refractive element, graded index lens, lens element with a different shape, e.g., a biconvex, convex/concave, plano-concave, or biconcave lens element, or any other suitable type of optic.) In this example, the focusing surface 212 is in the shape of a spherical cap, which is the region of a sphere that lies above (or below) a given plane, truncated on both sides by parallel planes (not shown) that are perpendicular to the circle subtended by the (untruncated) spherical cap. In this example, the parallel planes intersect the subtended circle along chords that are equidistant from and parallel to a diameter of the subtended circle. When viewed along the optical axis 202, the surface 212 is bounded by a perimeter 214 that has four edges—two straight edges parallel to the major axis 204 and two curved edges roughly parallel to the minor axis 206—arranged in a roughly rectangular shape that lacks circular symmetry about the optical axis 202.

The lens element 210 defines the device's major axis 204 and minor axis 206, which in turn affect the amount and orientation of the astigmatism correct provided by the device 200. For example, the lens element 210 may generate astigmatism in the direction orthogonal to the optical axis 202 and the major axis 204. In this case, the major axis 204 is perpendicular to the minor axis 206, which makes the implantable ophthalmic device 200 suitable for correcting regular astigmatism. The exact amount of astigmatism correction depends the refractive index of the lens element 210, the radius of curvature of the focusing surface 212, and on the ratio of the length of the major axis 204 to the length of the minor axis 206. The focusing surface 210 may have a radius of curvature of about 15 mm to about 100 mm. The device 200 may have a length along its major axis 204 of about 5.0 mm to about 7.0 nun and a length along its minor axis 206 of about 2.0 mm to about 6.0 mm for a length-to-width ratio of about 1.2 to about 3.5. In some examples, the amount of astigmatism may be from about 0.50 diopters to about 2.0 diopters.

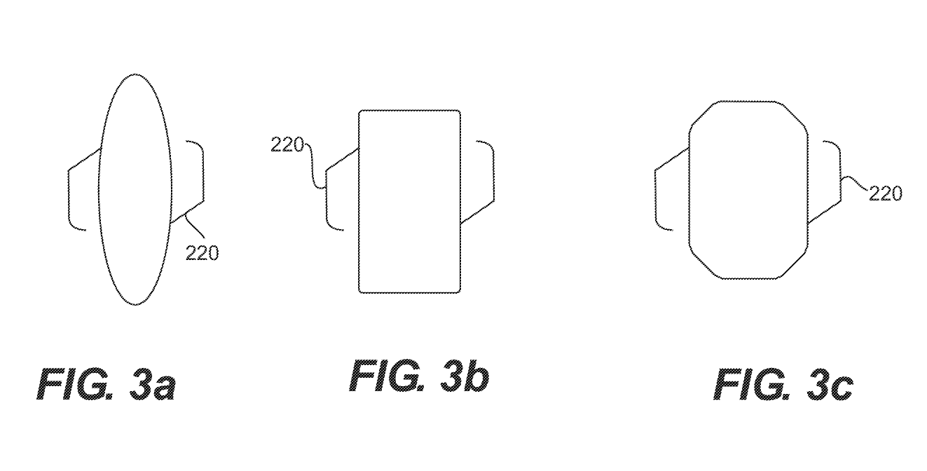

Those skilled in the art will readily appreciate that the perimeter 202 can be selected to have any suitable noncircular shape and/or combination or arrangement of edges. For example, as shown in FIGS. 3A-3C, the perimeter can be in the shape of ellipse, oval, rectangle, regular polygon, or irregular polygon, so long as the perimeter lacks circular symmetry. Note that the perimeter may have some degree of mirror and/or rotational symmetry about the optical axis. Such a non-circular optic may be provided either by shaping the perimeter accordingly, or by adding obscuration to a circular optic, causing the obscured zones of the optic to become opaque. The obscuration may be either permanent or dynamic, e.g., when provided by an electro-active cell comprising a liquid crystal that changes its optical absorbance upon stimulation by an electric potential.

Exemplary lens elements may be either conventional or non-conventional. A conventional lens corrects for conventional errors of the eye including lower order aberrations such as myopia, hyperopia, presbyopia, and regular astigmatism. A non-conventional lens corrects for non-conventional errors of the eye including higher order aberrations that can be caused by ocular layer irregularities or abnormalities. A spherical lens element may be a single-focus (monofocal) lens or a multifocal lens, such as a Progressive Addition Lens or a bifocal or trifocal lens.

Similarly, the focusing surface can be a section of an aspheric surface, which is a rotationally symmetric surface whose radius of curvature varies radially from its center. Aspheric surfaces used in lenses have shapes that have been traditionally defined by:

$$Z ( s ) = \frac{{Cs}^{2}}{1 + \sqrt{1 - ( 1 + k ) {C}^{2} {s}^{2}}} + {A}_{4} {s}^{4} + {A}_{6} {s}^{6} + {A}_{8} {s}^{8} + … , \qquad ( 1 )$$

where Z is the sag of the surface parallel to the optical axis, s is the radial distance from the optical axis, C is the curvature (i.e., the inverse of the radius), k is the conic constant, and Aₙ are weights for higher-order aspheric terms. When the aspheric coefficients are equal to zero, the resulting aspheric surface is considered to be a conic: for k=0, the conic surface is spherical (i.e., the lens is spherical rather than aspherical); for k>−1, the conic surface is ellipsoidal; for k=−1, the conic surface is paraboloidal; and for k=+1, it is hyperboloidal. The sag can also be described more precisely as

$$Z ( s ) = \frac{{C}_{bfs} {s}^{2}}{1 + \sqrt{1 - ( 1 + k ) {C}_{bfs}^{2} {s}^{2}}} + {u}^{4} {\sum }_{m = 0}^{M} {a}_{m} {Q}_{m}^{con} ( {u}^{2} ) , \qquad ( 2 )$$

where C_{bfs} is the curvature of the best-fit sphere, u=s/sₘₐₓ, Qₘ^{con} is the orthonormal basis of the asphere coefficients, and aₘ is a normalization term. In some cases, the aspheric surface may be shaped to provide spherical aberration that compensates for spherical aberration present in a patient's eye as described in PCT/US2011/038597 to Blum et al., which is incorporated herein by reference in its entirety.

Illustrative lens elements can be made of optical glass, plastic, thermoplastic resins, thermoset resins, a composite of glass and resin, or a composite of different optical grade resins or plastics. For example, lens elements can be made using injection-molded plastic or resin. Molten plastic is injected into an appropriately shaped mold and allowed to harden before being removed. Alternatively, the lens element can be made using conventional glass grinding and polishing techniques, and the other (optional) elements can be bonded or sealed together with the lens element.

Additional (optional) elements, such as an electro-active element, processor, sensor, and/or batteries may embedded in a plastic lens element during injection molding or affixed to a plastic lens element before the lens element has fully hardened. In some examples, the electro-active element is a liquid-crystal device that provides astigmatism correction as described below and/or increased depth of field as described in U.S. Pat. No. 7,926,940 to Blum et al., which is incorporated herein by reference in its entirety. If necessary, electronic components may be coated with an appropriate heat-resistant material to prevent damage during manufacturing. The position of the electro-active element with respect to the lens element can be adjusted during the molding process and may be chosen depending on each element's respective optical power. For example, the electro-active element can be positioned in the front, the center, or the rear of the lens element.

Lens elements (and the implantable ophthalmic device as a whole) can be flexible and/or have folding designs for easier implantation in the eye. For example, the lens element and device may fold about one or more fold lines for insertion, then unfold about the fold line(s) once properly positioned within the eye. Rigid components may be disposed on either side of the fold line(s) for ease of insertion. For more, see U.S. application Ser. No. 12/017,858, entitled “Flexible Electro-Active Lens,” and U.S. application Ser. No. 12/836,154, entitled “Folding Designs for Intraocular Lenses,” each of which is incorporated herein by reference in its entirety.

Alternatively, an exemplary implantable ophthalmic device may include a section of graded-index (GRIN) lens instead of or in addition to a focusing surface. A GRIN lens may be a cylindrical piece of glass, resin, plastic, or other suitable material whose refractive index varies as a function of radius, e.g., in the shape of a semicircle or parabola. A cylindrical or slab-like piece of GRIN material can be formed, cleaved, cut, ground, or otherwise shaped to provide the desired amount and orientation of astigmatism correction as described above.

Noncircular Apertures to Compensate Astigmatism

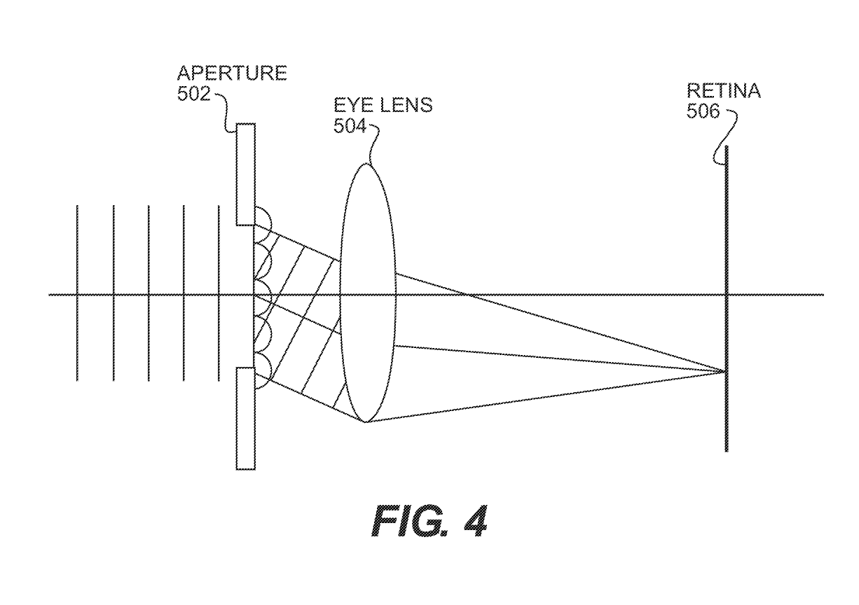

FIG. 4 illustrates how an aperture 502 can be used with an astigmatic lens, such as the eye's lens 504 (i.e., the cornea and/or the crystalline lens), to introduce compensatory astigmatism. Light (represented here as a plane wave) from an image at infinite remove propagates through the aperture 502 via the lens 504 to surface of the retina 506, which is in the focal plane of the lens 504. As understood by those of skill in the art, the aperture 502 can be treated as a virtual image at infinite distance whose shape is the spatial Fourier transform of the aperture 502 itself The lens 504 projects this virtual image onto the retina 506. The intensity detected by the retina is the square of the spatial Fourier transform of the aperture. Because the retinal image depends on the shape of the aperture, it is possible to compensate for retinal image aberrations, including astigmatism, by selecting an aperture shape that balances out or offsets blur induced by a misshapen cornea. For example, a noncircular (e.g., rectangular) aperture can be used to reduce the focused spot size in one dimension and/or increase the focused spot in another dimension to compensate for astigmatism.

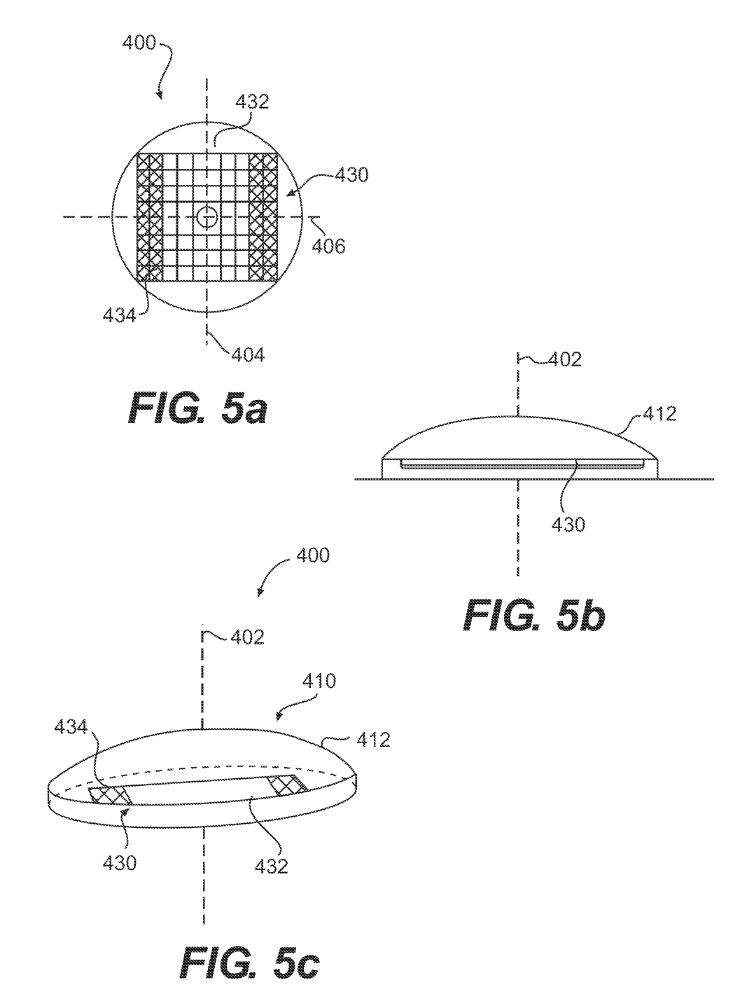

Noncircular apertures can be implemented with implantable ophthalmic devices that include electro-active elements and, optionally, optics with or without optical power. For example, FIGS. 5A-5C depict an implantable ophthalmic device 400 with a pixellated electro-active cell 430 that is embedded in a lens element 410 and configured to act as an aperture that occludes part of the lens element's focusing surface 412, which is orthogonal to the optical axis 402. The lens element 410 can be a conventional spherical lens, an aspheric lens, a GRIN lens, or a lens element with a circularly asymmetric lens similar to the lens element 210 shown in FIGS. 2A-2C. Alternatively, the electro-active cell 430 may be embedded in or affixed to an optic without any optical power.

As understood by those of skill in the art, the electro-active element 430 may comprise any suitable type of spatial-light modulator, such as a liquid crystal device. The electro-active cell 430 may be coupled to a processor (not shown) configured to actuate the processor in response to signals from a sensor, such as one or more photosensors configured to measure ambient light level, pupil diameter, object distance, etc. The device 400 may also include a battery, solar cell, or other power supply that powers the electro-active cell 430, the processor, and/or the sensor(s). For more details on electro-active elements, see, e.g., U.S. Pat. No. 7,926,940 to Blum et al., which is incorporated herein by reference in its entirety. More information on processors can be found in PCT/2011/040896 to Fehr et al., which is incorporated herein by reference in its entirety

Selectively actuating the pixels in the electro-active cell 430 causes some groups 434 of pixels to become opaque or reflective. Other pixels 432 remain at least partially transmissive to form an aperture with a circularly asymmetric perimeter. The exact selection of actuated pixels 434 (and hence the size, shape, and orientation of the aperture) can be adjusted as desired within the limits set by the number of pixels, the pixel pitch, and the pixel size. The orientation and length-to-width ratio of the aperture set the device's major axis 404 and minor axis 406 to provide a astigmatism correction of about 0.50 diopters to about 2.0 diopters, depending on the focusing surface's radius of curvature (e.g., 0-100 mm) and aperture length-to-width ration (e.g., 1.2-8.0).

Modeling the Performance of Noncircular Implantable Ophthalmic Devices

The performance of exemplary implantable noncircular ophthalmic devices can be described quantitatively by an optical transfer function (OTF), which is the complex contrast sensitivity function as a function of the spatial frequency of a target object. A complex contrast sensitivity function can be used to characterize the image quality because the optics of the eye may change the spatial frequency of the image relative to that of the target, depending on the target spatial frequency, in addition to reducing the contrast of the image. In principle, an OTF can be constructed for every object distance and illumination level. The OTF of the eye varies with object distance and illumination level, because both of these variables change the optics of the eye. The OTF of the eye may be reduced due to refractive errors of the eye, including astigmatism.

The image of a point object is the Fourier transform of the aperture convolved with the modulation transfer function (MTF) of the imaging optics, where the MTF is the real component of the OTF. The resulting point image is known as the point spread function (PSF), and may serve as an index of measurement of the quality of the ocular optic (i.e., a bare eye or eye corrected with a vision care means). The PSF of the retinal image is found to correlate with the quality of visual experience, especially when it is compromised by halos or glint or other image artifacts.

Calculating the MTF for models of perfect and astigmatic eyes with and without implantable noncircular apertures gives an indication of the efficacy of astigmatism treatment using noncircular implants (including devices with noncircular apertures and/or noncircular lens elements). In each case, it was assumed that the implantable noncircular aperture is simply an aperture at the capsular equator and has no optical power. The size of the aperture is taken to be 3.0 mm×5.0 mm. The image quality is computed for an object at infinity, and the MTF is calculated at 50 and 100 cycles per millimeter or line pairs per millimeter (cycles/mm or 1 p/mm) with entrance pupil diameters of 3 mm and 5 mm. The computed MTFs are “polychromatic” in that they are the weighted sum of MTFs at red, green, and blue wavelengths. Green is weighted twice as heavily as red, which is weighted equally to blue.

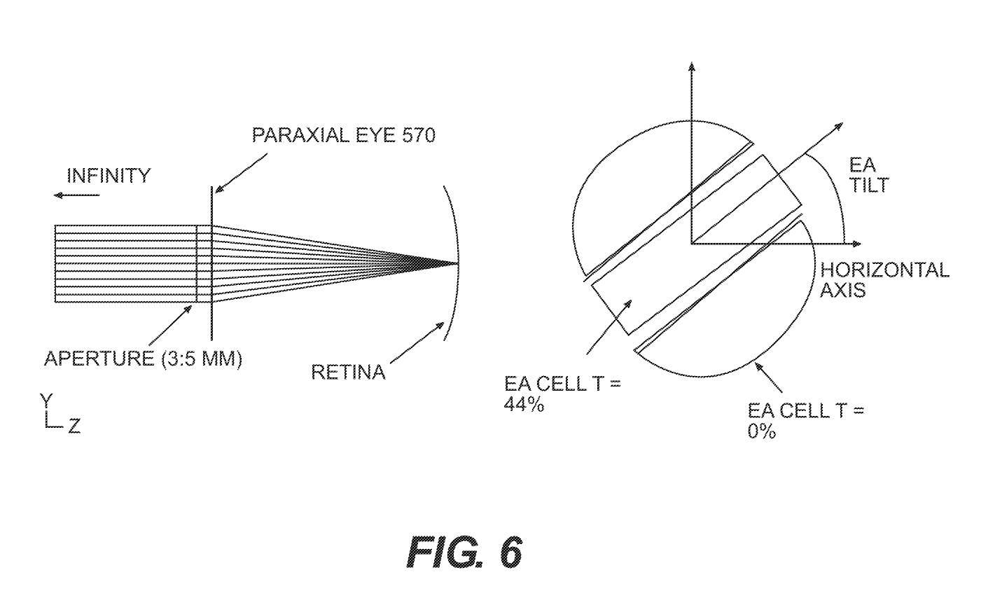

FIGS. 6-9 show a model and MTFs for a perfect eye (i.e., an eye that is not astigmatic) at different pupil diameters with and without an implantable noncircular aperture to show that the noncircular implant introduces astigmatism. The eye was modeled as a paraxial lens that focused light onto a curved plane representing the retina. The implantable noncircular aperture was modeled as a rectangular aperture (e.g., an actuated electro-active cell) with a width of 3 mm wide, a length of 5.8 mm, and a transmissivity of about 44%. The major (long) axis of the aperture was also taken to be tilted at various angles with respect to the vertical and horizontal axes of the eye.

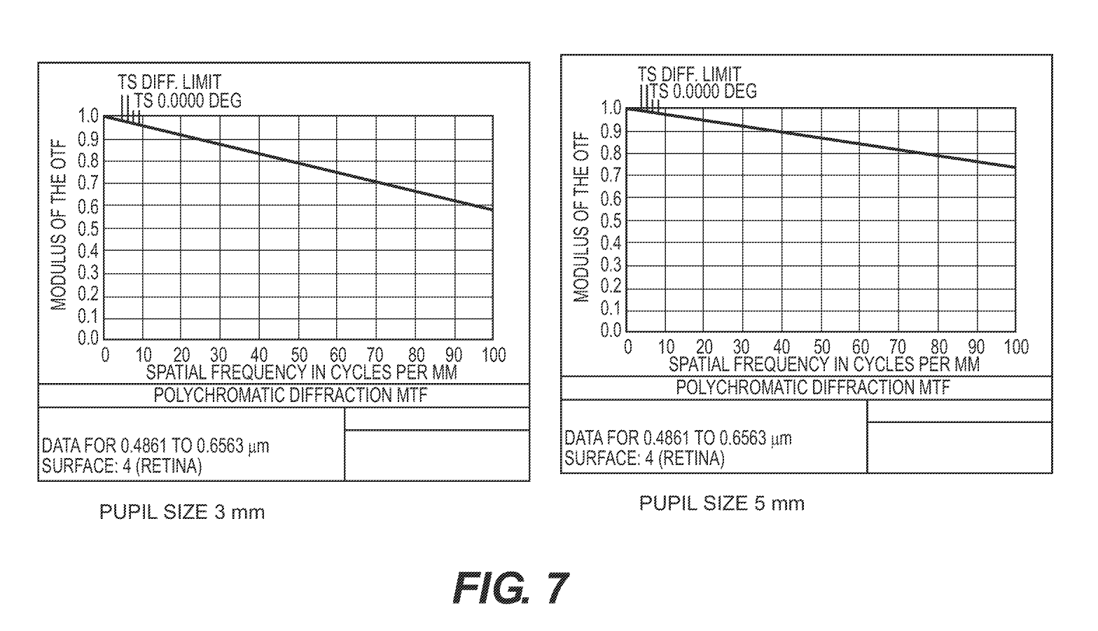

FIG. 7 shows plots of polychromatic MTFs (modulus of OTF) versus spatial frequency in cycles per millimeter for the perfect eye model at pupil diameters of 3 mm (left) and 5 mm (right). The perfect eye provides diffraction-limited vision at both pupil diameters; the MTF at 100 cycles/mm is 0.57 for the 3 mm pupil diameter and 0.74 for the 5 mm pupil diameter. There is no astigmatism; the tangential and sagittal MTFs are identical.

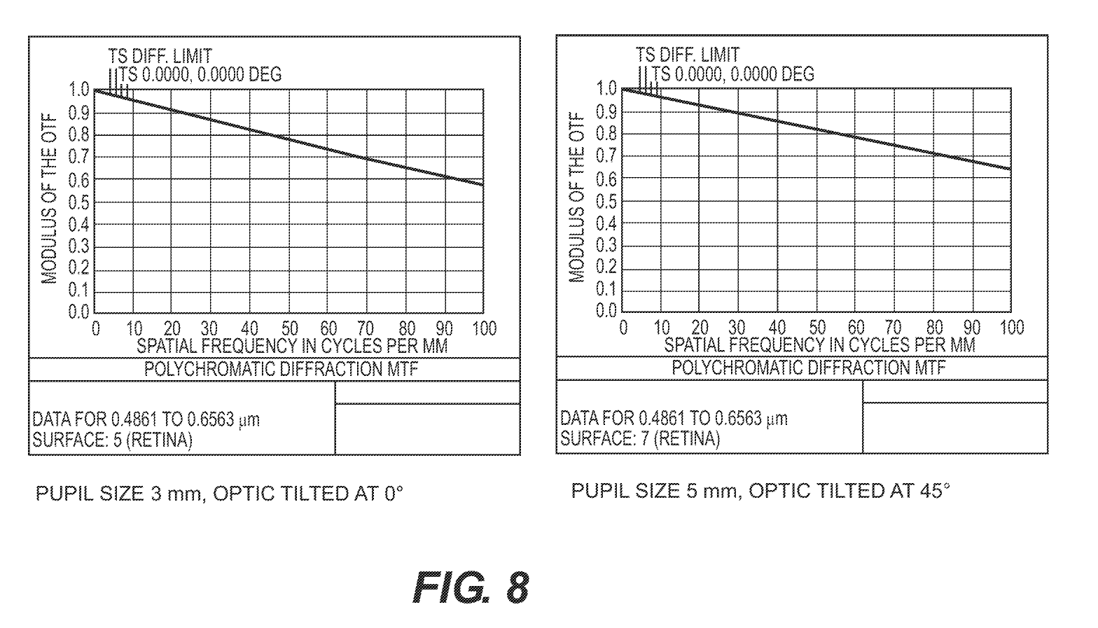

FIG. 8 shows plots of polychromatic MTFs versus spatial frequency for the perfect eye model with an implanted noncircular optic at pupil diameters of 3 mm (left) and 5 mm (right). The MTFs for the 3 mm pupil diameter with and without the implantable noncircular aperture are identical. Increasing the pupil diameter to 5 mm decreases the MTF to 0.65 with negligible astigmatism for a 5 mm pupil diameter when the noncircular optic is tilted by 45°.

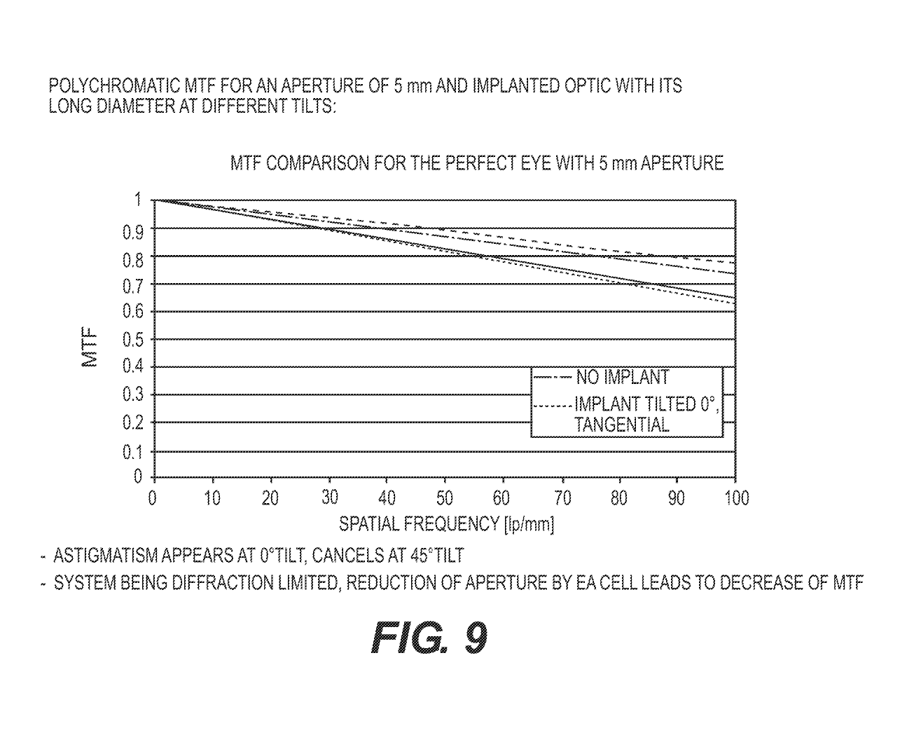

FIG. 9 is a plot of polychromatic MTFs versus spatial frequency computed with the perfect eye model at a pupil diameter of 5 mm for different orientations of the implantable noncircular aperture. The dashed line indicates the diffraction-limited MTF. In diffraction-limited systems, reducing the size of the aperture (e.g., by actuating an electro-active cell that acts as the aperture) decreases the MTF. Aligning the long axis of the implantable noncircular aperture with the eye's vertical axis causes astigmatism to appear. Rotating the implantable noncircular aperture to a 45° tilt angle causes the astigmatism to disappear in sagittal and tangential MTF projections. FIG. 9 shows that changing the size and orientation of aperture occluding a lens in an optical system causes the astigmatism of the optical system to change.

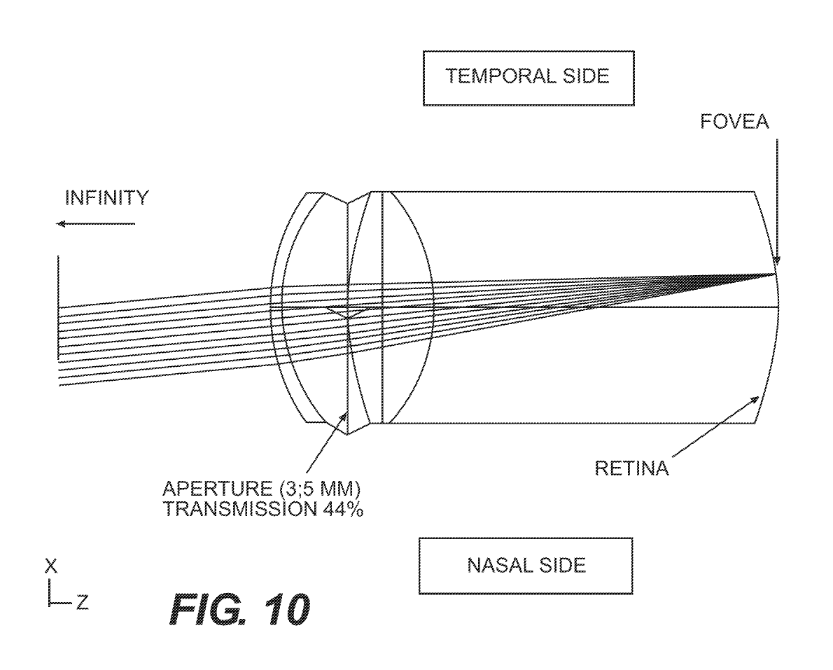

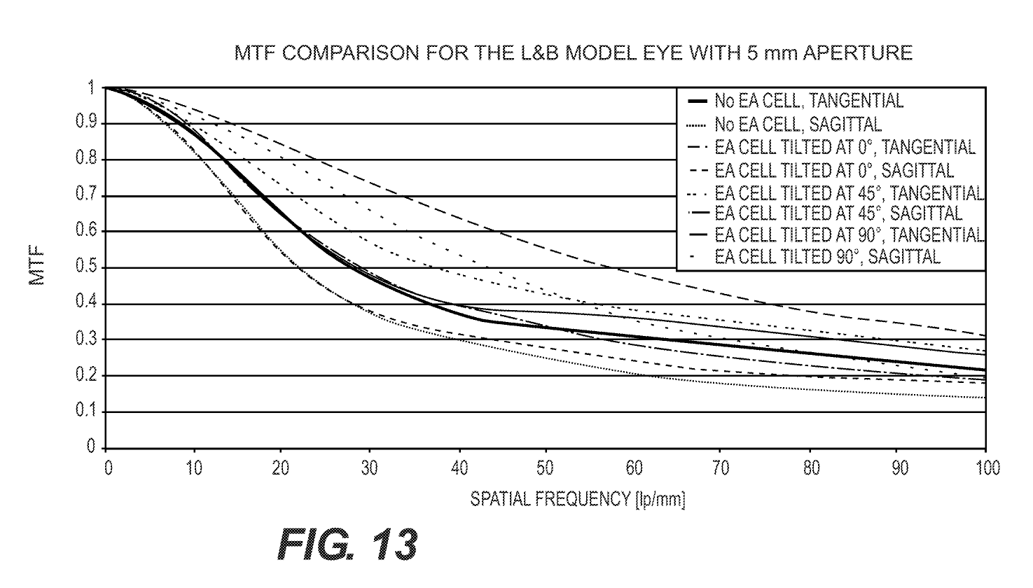

FIGS. 10-14 show a model of an astigmatic eye and polychromatic MTFs for the astigmatic eye with and without implanted noncircular optics. As shown in FIG. 10, the astigmatic eye is modeled as a real eye—in this case, using the well-known Liou and Brennan eye model—that images an object at infinity aligned onto fovea. The principal ray emanating from the object is tilted at 5° with respect to the optical axis. A homogeneous electro-active element is used to model a rectangular clear aperture with a transmissivity of 44%.

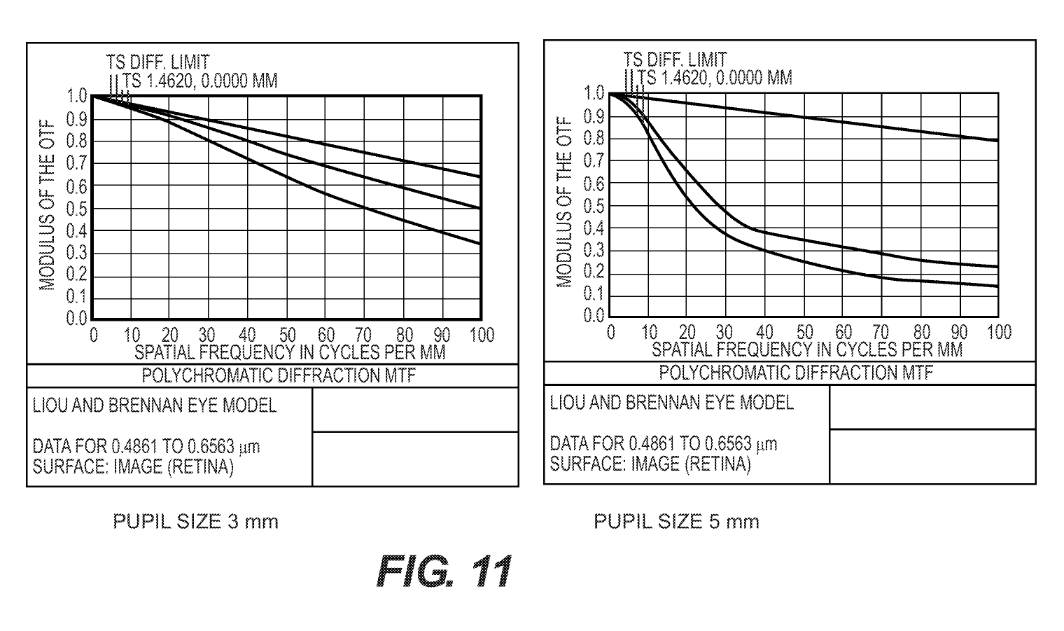

FIG. 11 shows plots of polychromatic MTFs computed using the Liou and Brennan model for pupil diameters of 3 mm (left) and 5 mm (right). The average MTF is much lower than with the perfect eye: at a spatial frequency of 100 cycles/mm, the average modulus of the OTF is 0.42 for the 3 mm pupil diameter and 0.18 for an aperture of 5 mm pupil diameter. The separation of sagittal (top) and tangential (bottom) MTFs indicates the presence of astigmatism, which limits the retinal image quality.

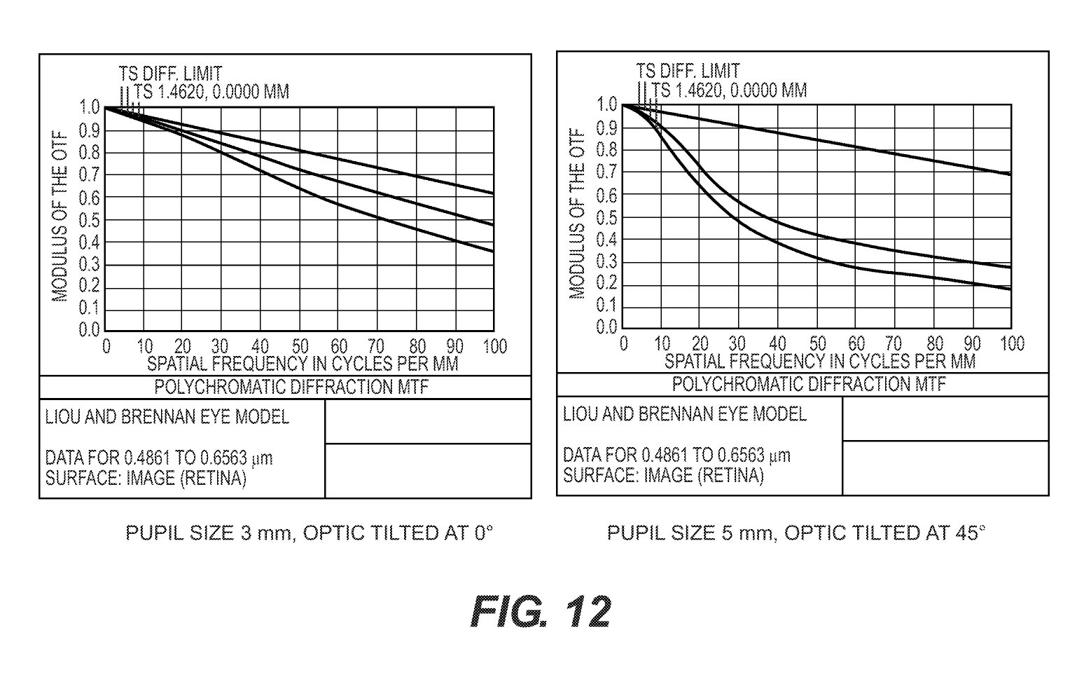

FIG. 12 shows plots of polychromatic MTFs computed using the Liou and Brennan model for an astigmatic eye with a 3 mm×5.8 mm aperture and different pupil diameters and major axis orientations. The plot at left shows that placing an implantable noncircular aperture tilted at 0° in an eye with a pupil diameter of about 3 mm has negligible impact on astigmatism. For 5 mm pupil diameter, however, implanting the noncircular aperture with a tilt of 45° improves the MTF by about 0.1 beyond spatial frequencies of 27 cycles/mm. The difference in modulus remains about same despite the increase in modulus at higher spatial frequency.

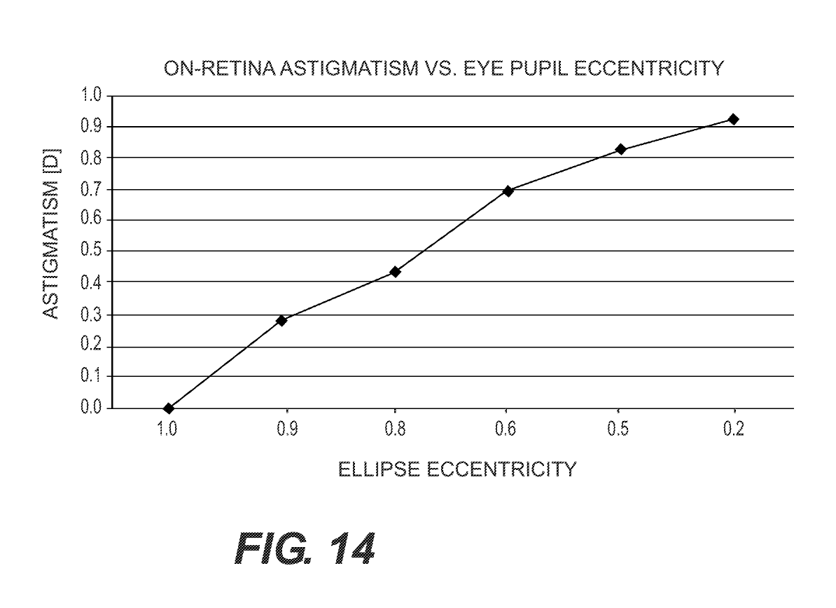

FIG. 14 is a plot of polychromatic MTFs versus spatial frequency computed with the Liou and Brennan eye model at a pupil diameter of 5 mm for different orientations of the 3 Mm×5.8 mm of FIGS. 10 and 12. Dashed lines represent sagittal MTFs and solid lines represent tangential MTFs. (Black solid and dashed lines indicate MTFs without implanted optics.) The average MTF at 100 cycles/mm is 0.25 for a 0° tilt, 0.23 for 45° tilt and 0.23 for 90° tilt vents 0.18 for the eye without an aperture. For an aperture tilted by 90°, the astigmatism is null at 60 cycles/mm. Increases the length-to-width ration of the aperture decreases aberrations and leads to an increase of MTF

FIG. 15 is a plot of on-retinal astigmatism versus the eccentricity of an elliptically shaped optic of an implant. Eccentricity is defined as the ratio of the minor axis length to major axis length. An eccentricity of 1.0 signifies a circular optic, consistent with zero astigmatism. The magnitude of induced compensatory astigmatism increases monotonically as the eccentricity deviates more and more from 1.0, as shown in FIG. 15. At an eccentricity of about 0.2 (e.g., an aperture of 1 mm×5 mm), the astigmatism increases to about 1.0 diopters.

Implanting Noncircular Implantable Ophthalmic Devices

Illustrative noncircular implantable ophthalmic devices may be implanted using modified versions of standard surgical techniques to treat common ophthalmological disorders, including cataracts. For example, an illustrative rectangular lens optic may be used to correct for corneal astigmatism in cataract patients (as well as to treat replace the crystalline lens affected by the cataract). Before implanting the noncircular device, an ophthalmologist may determine the type and amount of corneal astigmatism, e.g., by using a keratometer or keratoscope to confirm the presence of astigmatism and to measure the curvature of the cornea.

Once the ophthalmologist has determined the type and degree of astigmatism, he marks the patient's scelera (reference numeral 110 in FIG. 1) or other anatomical feature with ink or any other suitable marking substance to indicate the orientation of at least one of the principal meridians. For example, he may inscribe a fiducial mark, such as a vertical line, on the scelera to indicate with-the-rule regular corneal astigmatism. Next, he inserts or implants the noncircular implant into a suitable part of the patient's eye, such as the anterior chamber or posterior chamber of the eye, into the capsular sac, or the stroma of the cornea (similar to a corneal inlay), or into the epithelial layer of the cornea (similar to a corneal onlay), or within any anatomical structure of the eye. He aligns the major axis and/or minor axis of the noncircular implant with the fiducial mark, then secures the noncircular implant in position, e.g., by deploying lens anchors. Best results may be obtained when the long diameter of the implant is orthogonal to the axis of corneal astigmatism. Those of skill in art will appreciate that alignment need not be a separate step; i.e., the noncircular implant can be inserted or implanted with the desired alignment.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. An implantable ophthalmic device comprising:
an optic having an optical axis, the optic having a surface that is perpendicular to the optical axis and bounded by a perimeter that lacks circular symmetry.

2. The implantable ophthalmic device of claim 1 wherein the optic is a lens element.

3. The implantable ophthalmic device of claim 1 wherein the optic has an astigmatism of about 0.50 diopters to about 2.0 diopters.

4. The implantable ophthalmic device of claim 1 wherein the optic has at least one major axis perpendicular to the optical axis and at least one minor axis perpendicular to the major axis.

5. The implantable ophthalmic device of claim 4 wherein the at least one major axis has a length of about 4 mm to about 7 mm.

6. The implantable ophthalmic device of claim 4 wherein the optic is configured such that, when implanted in an eye with corneal astigmatism, the at least one major axis is orthogonal to an axis of the corneal astigmatism.

7. The implantable ophthalmic device of claim 1 wherein the optic has a spherical aberration.

8. The implantable ophthalmic device of claim 1 wherein the optic has a graded index profile.

9. The implantable ophthalmic device of claim 1 wherein the surface includes a section of a sphere.

10. The implantable ophthalmic device of claim 1 wherein the surface includes a section of an aspheric surface.

11. The implantable ophthalmic device of claim 1 wherein the surface has a radius of curvature of about 15 mm to about 100 mm.

12. The implantable ophthalmic device of claim 1 wherein the perimeter includes a plurality of edges.

13. The implantable ophthalmic device of claim 12 wherein said optic has a perimeter that defines a convex polygon.

14. The implantable ophthalmic device of claim 12 wherein the plurality of edges forms a rectangle.

15. The implantable ophthalmic device of claim 12 wherein the at least one pair of edges has a ratio of width to length of about 1.2 to about 3.5.

16. The implantable ophthalmic device of claim 1 wherein the perimeter forms an ellipse.

17. The implantable ophthalmic device of claim 16 wherein the ellipse has a major axis with a length of about 5.0 mm to about 7.0 mm and a minor axis with a length of about 2.0 mm to about 6.0 mm.

18. The implantable ophthalmic device of claim 1 further comprising:
at least one lens anchor coupled to the optic and configured to maintain the optic in a stable position when implanted in a patient's eye.

19. An implantable ophthalmic device comprising:
an electro-active cell configured to form a noncircular aperture, in a plane normal to the optical axis, whose major axis is aligned with respect to a principal meridian of an astigmatic eye.

20. The implantable ophthalmic device of claim 19 wherein the noncircular aperture introduces an astigmatism of about 0.10 diopters to about 2.0 diopters.

21. The implantable ophthalmic device of claim 19 wherein the major axis is orthogonal to the principal meridian of the astigmatic eye.

22. The implantable ophthalmic device of claim 19 wherein the major axis has a length that is about 1.2 to about 3.5 times larger than the shortest dimension of the noncircular aperture.

23. A method of correcting astigmatism, the method comprising:
actuating an electro-active cell implanted in an astigmatic eye to form a noncircular aperture, in a plane normal to the optical axis, whose major axis is aligned with respect to a principal meridian of an astigmatic eye.

24. The method of claim 23 wherein actuating the electro-active cell to form the noncircular aperture introduces an astigmatism of about 0.10 diopters to about 2.0 diopters.

25. The method of claim 23 wherein actuating the electro-active cell to form an aperture includes forming the major axis orthogonal to an axis of corneal astigmatism.

26. The method of claim 23 wherein the major axis has a length that is about 1.2 to about 3.5 times larger than the shortest dimension of the noncircular aperture.

27. A method of implanting an optic in an eye, the optic having a surface that is perpendicular to the optical axis of the eye and bounded by a perimeter that lacks circular symmetry, the method comprising:
(a) making a mark on the scelera of the eye indicative of an astigmatic characteristic of the eye;
(b) inserting the optic into the eye; and
(c) aligning the perimeter of the optic with respect to the mark on the scelera.

28. The method of claim 23 further comprising:
measuring the astigmatic characteristic of the eye.

29. The method of claim 23 further comprising:
deploying a lens anchor coupled to the optic after the perimeter of the optic is aligned with respect to the mark on scelera.
